(19) United States
(12) Patent Application Publication
(10) Pub. No.: US 2024/0224996 A1
(43) Pub. Date: Jul. 11, 2024

(54) LIQUID CHEMICAL STERILIZATION CHEMISTRY

(71) Applicant: Medivators Inc., Minneapolis, MN (US)

(72) Inventors: Huyen Bui, Minneapolis, MN (US); Nam Bui, Minneapolis, MN (US); Kristopher Murphy, Minneapolis, MN (US); Tuan Nguyen, Minneapolis, MN (US); Elaine Ferreira Vrieze, Minneapolis, MN (US)

(21) Appl. No.: 18/558,394

(22) PCT Filed: May 5, 2022
(86) PCT No.: PCT/US22/72126

§ 371 Date: Nov. 1, 2023

Related U.S. Application Data
(60) Provisional application No. 63/186,429, filed on May 10, 2021.

(51) Int. Cl. A01N 37/02; A01N 25/02; A01N 25/22; A01N 59/00; A01P 1/00

(52) U.S. Cl. CPC A01N 37/02; A01N 25/02; A01N 25/22; A01N 59/00; A01P 1/00

(57) ABSTRACT

The invention relates generally to a two part liquid composition that includes components (A) and (B), when mixed yield an aqueous disinfecting composition. Component (A) includes hydrogen peroxide, acetic acid and/or peracetic acid and a stabilizer. Component (B) includes a buffer, an anticorrosive agent, a solubilizer and a cleaner. Combining component (A) and component (B) provides a liquid sterilant.

### CROSS REFERENCE TO RELATED APPLICATIONS

This application claims priority to and the benefit of U.S. Provisional Application with Ser. No. 63/186,429, filed on May 10, 2021, entitled LIQUID CHEMICAL STERILIZATION CHEMISTRY, which is herein incorporated by reference in its entirety.

### FIELD OF THE INVENTION

The invention relates generally to a two part liquid composition that includes components (A) and (B), when mixed yield an aqueous disinfecting composition. Component (A) includes hydrogen peroxide, acetic acid and/or peracetic acid and a stabilizer. Component (B) includes a buffer, an anticorrosive agent, a solubilizer and a cleaner. Combining component (A) and component (B) provides a liquid sterilant.

### BACKGROUND OF THE INVENTION

A perfect disinfectant would offer complete and full microbiological sterilization, without harming humans and useful forms of life, be inexpensive, and non-corrosive. However, ideal disinfectants do not exist. Most disinfectants are also, by nature, potentially harmful (even toxic) to humans or animals.

The choice of disinfectant to be used depends on the particular situation. Some disinfectants have a wide spectrum (kill many different types of microorganisms), while others kill a smaller range of disease-causing organisms but are preferred for other properties (they may be non-corrosive, non-toxic, or inexpensive). Peracetic acid and hydrogen peroxide compositions have been used to disinfect various surfaces including surfaces of instruments. However, contamination of the peracetic acid/hydrogen peroxide composition is commonplace by a user. Contamination of the peracetic acid/hydrogen peroxide composition causes degradation and instability of the composition.

Current processing conditions with peracetic acid solutions to clean and/or disinfect medical devices, such as catheters, endoscopes and the like, are generally conducted at elevated temperature ranges from about 50° C. up to 100° C. These caustic conditions are harsh on the medical device and can lead to degradation of the device which decreases the use lifespan of the device.

Therefore, a need exists for sterilization compositions that overcome one or more of the current disadvantages noted above.

### BRIEF SUMMARY OF THE INVENTION

The present embodiments surprisingly provide a mild method to cleanse, disinfect and/or sterilize medical devices that are soiled with bodily fluids and/or waste. The mild cleansing conditions include with a two part to a two part liquid composition that includes components (A) and (B), when mixed yield an aqueous disinfecting/sterilization composition. Component (A) includes hydrogen peroxide, acetic acid and/or peracetic acid and a stabilizer. Component (B) includes a buffer, an anticorrosive agent, a solubilizer and a cleaner. Combining component (A) and component (B) provides a liquid sterilant which is used to treat the soiled medical device or surface. The cleansing conditions are effective at ambient temperature, or at elevated temperatures of from about 20° C. to about 50° C. Bacteria, microorganisms and viruses, for example, are eliminated upon treatment with the sterilant and the surface or medical device is rendered disinfected and/or sterilized.

The present embodiments also provide for kits that include: (a) enclosed containers that include removable closures; (b) the components (A) and (B) of the liquid sterilant as described herein, located inside the enclosed containers, and (c) printed indicia located on the enclosed containers.

The present embodiments also provide for a method of reducing the number of microbes located upon a substrate. In some embodiments, the method includes contacting the substrate with an effective amount of the liquid sterilant described herein, for a sufficient period of time, effective to reduce the number of microbes located upon the substrate.

The present embodiments also provide for a method of disinfecting or sterilizing a substrate. In some embodiments, the method includes contacting the substrate with an effective amount of the liquid sterilant described herein, for a sufficient period of time, effective to disinfect or sterilize the substrate. The present embodiments also provide for a method of disinfecting or sterilizing a medical device. In some embodiments, a method of disinfecting or sterilizing an endoscopic device is achieved with the use of the liquid sterilant described herein.

Surprisingly, it was found that the combination of a solubilizer and a cleaner had improved cleansing effects and anticorrosive properties than when one of the components was not present in the ultimate liquid sterilant.

While multiple embodiments are disclosed, still other embodiments of the present will become apparent to those skilled in the art from the following detailed description. As will be apparent, the invention is capable of modifications in various obvious aspects, all without departing from the spirit and scope of the present invention. Accordingly, the detailed descriptions are to be regarded as illustrative in nature and not restrictive.

### BRIEF DESCRIPTION OF THE DRAWINGS

FIG. 1 shows the PAA stability data for the stability study of Part A.

FIG. 2 shows the hydrogen peroxide stability data for the stability study of Part A.

FIG. 3 shows the acetic acid stability data for the stability study of Part A.

### DETAILED DESCRIPTION

In the specification and in the claims, the terms “including” and “comprising” are open-ended terms and should be interpreted to mean “including, but not limited to . . . .” These terms encompass the more restrictive terms “consisting essentially of” and “consisting of.”

It must be noted that as used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprising”, “including”, “characterized by” and “having” can be used interchangeably.

Unless defined otherwise, all technical and scientific terms used herein have the same meanings as commonly understood by one of ordinary skill in the art to which this invention belongs. All publications and patents specifically mentioned herein are incorporated by reference in their entirety for all purposes including describing and disclosing the chemicals, instruments, statistical analyses and methodologies which are reported in the publications which might be used in connection with the invention. All references cited in this specification are to be taken as indicative of the level of skill in the art. Nothing herein is to be construed as an admission that the invention is not entitled to antedate such disclosure by virtue of prior invention.

Described herein are two part liquid compositions that include components (A) and (B). When components (A) and (B) are combined, they provide a liquid disinfecting/sterilization composition termed “liquid sterilant”. Component (A) of the liquid sterilant includes hydrogen peroxide, acetic acid and/or peracetic acid and a stabilizer. Component (B) of the liquid sterilant includes a buffer, an anticorrosive agent, a solubilizer and a cleaner. Combining component (A) and component (B) provides the liquid sterilant which is used to treat soiled medical devices or surfaces. The cleansing conditions are effective at ambient temperature, or at elevated temperatures of from about 20° C. to about 50° C.

Values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” should be interpreted to include not only the explicitly recited amount of about 0.1 wt. % to about 5 wt. %, but also the individual concentrations (e.g., 1%, 2%, 3%, and 4%) and the sub-ranges (e.g., 0.5%, 1.1%, 2.2%, 3.3%, and 4.4%) within the indicated range.

When describing the present embodiments, the following terms have the following meanings, unless otherwise indicated.

The term “about” can allow for a degree of variability in a value or range, for example, within 10%, within 5%, or within 1% of a stated value or of a stated limit of a range.

The term “room temperature” as used herein refers to a temperature of about 15° C. to 28° C.

The term “hydrogen peroxide” or “H₂O₂” refers to the compound chemically designated as dihydrogen dioxide, having the CAS Reg. No. 7722-84-1. In specific embodiments of the s, the hydrogen peroxide includes water. In further specific embodiments, the hydrogen peroxide is 50% wt. % hydrogen peroxide in water. The hydrogen peroxide can be present in the composition, in any suitable and effective amount.

The term “organic acid” refers to an organic compound with acidic properties. The most common organic acids are the carboxylic acids, whose acidity is associated with their carboxyl group —COOH. Sulfonic acids, containing the group —SO₂OH, are relatively stronger acids. The relative stability of the conjugate base of the acid determines its acidity. Other groups can also confer acidity, usually weakly: —OH, —SH, the enol group, and the phenol group. Organic compounds containing these groups are generally referred to as organic acids. An example of an organic acid is acetic acid.

The term “acetic acid” or “ethanoic acid” refers to an organic compound with the chemical formula CH₃CO₂H (also written as CH₃COOH), having the CAS Reg. No. 64-19-7.

The term “glacial acetic acid” refers to undiluted and relatively concentrated, water-free (anhydrous) acetic acid

The term “ethylenediaminetetraacetic acid” (EDTA) (2,2′,2″,2′″-(ethane-1,2-diyldinitrilo)tetraacetic acid)(CAS Number 60-00-4; 63819-2-6 dihydrate disodium salt), is known in the art as a chelating agent. In some embodiments, the compositions described herein do not include EDTA.

The term “chelator,” “chelant” or “chelating agent” refers to a compound that forms soluble, complex molecules with certain metal ions, inactivating the metal ions (or to some extent, countering the effects of the metal ions), so that they cannot normally react with other compounds, elements or ions. In specific embodiments, the chelator effectively chelates transition metals. One suitable type of chelator is/are sulfonic acids, more particularly, polymers or solid supports which contain sulfonic acid functionality. In specific embodiments, the chelator will effectively chelate any transition metals and/or alkaline earth metals present in any of the components of the liquid sterilant.

In particular, the chelator can be a sulfonic acid group that is incorporated into a polymer. For example, the polymer can be styrene based that is functionalized with sulfonic acid groups. The styrenic polymer can be a copolymer, such as styrene/divinylbenzene. The polymer may further be crosslinked. Examples of commercially available sulfonic acid functionalized polymers include those such as Dowex® 50WX4-200, Dowex® DR2030, Amberlite IR120 Na, Amberlite IRN99, Amberlyst 15 hydrogen (CAS Number 39389-20-3) and Amberlite strong acidic cation exchange sodium form available from Dow Chemical Company, which are styrene-divinylbenzene copolymers.

Alternatively, a copolymer of tetrafluoroethylene (TFE) and Sulfonyl Fluoride Vinyl Ether (SFVE) F₂C═CF—O—CF₂CF₂—SO₂F is a useful material. Aquivion® PFSA (perfluorosulfonic acid) ionomers, available from Solvay, are based on this copolymer or tetrafluoroethylene-perfluoro(3-oxa-4-pentenesulfonic acid) copolymers (e.g., [CF₂CF(OCF₂CF₂SO₃H)]ₘ[CF₂CF₂]ₙ, as Aquivion E98-15S, Aquivion E98-09S, Aquivion PW79S, or Aquivion E87-05S available from Sigma or Krackeler Scientific, Inc.). and are available in a membrane, as a powder, in a dispersion or as pellets. These are all perfluorosulfonic acid resins.

In one aspect, the perfluorosulfonic acid pellets can be extruded/coextruded with other polymers to form films or shaped into a container to hold the remaining components of the embodiments. Suitable extrusion polymers include, for example, polyethylenes, e.g., (high density polyethylene, HDPE) and polypropylenes.

In another embodiment, the polymer can be derived from 2-acrylamido-2-methylpropane sulfonic acid (AMPS). Additionally, AMPS can be used to coat the lining of a container and then be polymerized to the surface of the container as a protective/chelating coating.

It should be understood that the requisite sulfonic acid group may need to be first treated with an acidic solution to provide the free acid as necessary.

The polymeric resin chelator can be added to the liquid sterilants described herein. Alternatively, components (A) and (B) of the liquid sterilant can be passed through the polymeric resin chelator. In another embodiment, the polymeric resin chelator can be in the form of a membrane and the membrane is in contact and remains in contact with the composition. In still another embodiment, the polymeric resin chelator is incorporated into a container which hold the compositions described herein. In certain embodiments, the polymer resin chelator is coated onto the interior of a container that is used to store the compositions described herein. In still another embodiment, the polymeric chelator can be placed within a “mesh pouch” or other containment system that can be placed into a container with the compositions described herein.

One advantage of utilizing the polymeric resin chelator is that users of the compositions often contaminate the composition in between uses. That is, an individual may place a used wipe, sponge, or rag, medical device, instrument, etc. against or within the container that houses the composition, thus transferring contaminants to the container. The polymeric resin chelators described herein help to stabilize the peracetic acid/hydrogen peroxide compositions by complexing with/removing the undesired contaminants, such as metal ions.

It should be understood that one advantage of the polymeric resin chelator is that it does not dissolve in the embodiments described herein. That is, the polymer resin remains in the solution but does not become homogeneous with the remaining components. Not to be limited by theory, it is believed that the polymeric resin chelator provides surface contact with the components of the composition and removes metallic contaminants from the solution to stabilize the composition. As a result, the components of the composition, e.g., the hydrogen peroxide and/or the peracetic acid, do not degrade over time due to metallic components. Additionally, the polymeric resin chelator does not cause a residue to remain on a treated surface after the surface has been treated with the compositions described herein.

The term “anticorrosive agent” or “corrosion inhibitor” refers to a compound that, when added to a liquid or gas, decreases the corrosion rate of a material, typically a metal or an alloy. Suitable anticorrosive agents include, e.g., benzotriazole and sodium dodecyl sulfate (SDS).

The term “benzotriazole” or “BTA” refers to the compound 1H-benzotriazole or 1,2,3-benzotriazole, having the CAS Reg. No. 95-14-7.

The term “surfactant” refers to a compound capable of lowering the surface tension of a liquid, the interfacial tension between two liquids, or that between a liquid and a solid. Surfactants may act as detergents, wetting agents, emulsifiers, foaming agents, and/or dispersants. The surfactant can be non-ionic, anionic or cationic. Additionally, the surfactant can include one or more non-ionic surfactants, one or more anionic surfactants, and/or one or more cationic surfactants.

The term “non-ionic surfactant” or “nonionic surfactant” refers to a surfactant, in which the total number of electrons is equal to the total number of protons, giving it a net neutral or zero electrical charge. One suitable class of non-ionic surfactants includes the Pluronic® poloxamers.

Poloxamers are nonionic triblock copolymers composed of a central hydrophobic chain of polyoxypropylene (poly(propylene oxide)) flanked by two hydrophilic chains of polyoxyethylene (poly(ethylene oxide)). Poloxamers are also known by the trade name Pluronics*.

Because the lengths of the polymer blocks can be customized, many different poloxamers exist, that have slightly different properties. For the generic term “poloxamer,” these copolymers are commonly named with the letter “P” (for poloxamer) followed by three digits, the first two digits “×” (times) 100 give the approximate molecular mass of the polyoxypropylene core, and the last digit×10 gives the percentage polyoxyethylene content (e.g., P407=Poloxamer with a polyoxypropylene molecular mass of 4,000 g/mol and a 70% polyoxyethylene content). For the Pluronic® tradename, coding of these copolymers starts with a letter to define its physical form at room temperature (L=liquid, P=paste, F=flake (solid)) followed by two or three digits. The first digit (two digits in a three-digit number) in the numerical designation, multiplied by 300, indicates the approximate molecular weight of the hydrophobe; and the last digit×10 gives the percentage polyoxyethylene content (e.g., L61=Pluronic with a polyoxypropylene molecular mass of 1,800 g/mol and a 10% polyoxyethylene content). In the example given, poloxamer 181 (P181)=Pluronic L61.

The term “Pluronic® 10R5 surfactant block copolymer” refers to polyoxypropylene-polyoxyethylene block copolymer, having the CAS Reg. No. 9003-11-6.

Other nonionic surfactants include, but are not limited to, fatty alcohols, polyoxyethylene glycol alkyl ethers (Brij), polyoxypropylene glycol alkyl ethers, glucoside alkyl ethers, polyoxyethylene glycol octylphenol ethers, polyoxyethylene glycol alkylphenol ethers, glycerol alkyl esters, polyoxyethylene glycol sorbitan alkyl esters, sorbitan alkyl esters, cocamide MEAs, cocamide DEAs, dodecyl dimethylamine oxides, block copolymers of polyethylene glycol and polypropylene glycols.

Suitable fatty alcohols include, but are not limited to, cetyl alcohol, stearyl alcohol, cetostearyl alcohol (consisting predominantly of cetyl and stearyl alcohols) and oleyl alcohol.

Suitable polyoxyethylene glycol alkyl ethers, include but are not limited to (Brij), for example CH₃—(CH₂)₁₀₋₁₆—(O—C₂H₄)₁₋₂₅—OH, or octaethylene glycol monododecyl ether or pentaethylene glycol monododecyl ether.

Suitable polyoxypropylene glycol alkyl ethers include CH₃—(CH₂)₁₀₋₁₆—(O—C₃H₆)₁₋₂₅—OH.

Suitable glucoside alkyl ethers include CH₃—(CH₂)₁₀₋₁₆—(O-Glucoside)₁₋₃-OH, and, for example, include decyl glucoside, lauryl glucoside, and octyl glucoside.

Suitable polyoxyethylene glycol octylphenol ethers include C₈H₁₇—(C₆H₄)—(O—C₂H₄)₁₋₂₅—OH. One exemplary material is TRITON X-100.

Suitable polyoxyethylene glycol alkylphenol ethers include C₉H₁₉-(C₆H₄)—(O—C₂H₄)₁₋₂₅—OH. One example is Nonoxynol-9.

In one aspect, a suitable glycerol alkyl ester is glyceryl laurate.

In another aspect, a suitable polyoxyethylene glycol sorbitan alkyl ester is polysorbate.

In still another aspect, suitable sorbitan alkyl esters are referred to as SPAN, e.g., SPAN-20, sorbitan monolaurate.

The term “cationic surfactant” refers to a surfactant, in which the total number of electrons is less than the total number of protons, giving it a net positive electrical charge.

One kind of cationic surfactant is typically based on pH-dependent primary, secondary or tertiary amines. The primary amines become positively charged at a pH<10, and the secondary amines become charged at a pH<4. One example is octenidine dihydrochloride.

Another type of cationic surfactant is based on permanently charged quaternary ammonium cations, such as alkyltrimethylammonium salts. These include but are not limited to cetyl trimethylammonium bromide (CTAB), hexadecyl trimethyl ammonium bromide, cetyl trimethylammonium chloride (CTAC), cetylpyridinium chloride (CPC), polyethoxylated tallow amine (POEA), benzalkonium chloride (BAC), benzethonium chloride (BZT), 5-Bromo-5-nitro-1,3-dioxane, dimethyldioctadecylammonium chloride and dioctadecyldimethylammonium bromide (DODAB).

The term “anionic surfactant” refers to a surfactant in which the total number of electrons is greater than the total number of protons, giving it a net negative electrical charge. One suitable anionic surfactant is sodium lauryl sulfate.

Anionic surfactants have a permanent anion, such as a sulfate, sulfonate or phosphate anion associated with the surfactant or has a pH-dependent anion, for example, a carboxylate.

Sulfates can be alkyl sulfate or alkyl ether sulfates.

Suitable alkyl sulfates include, but are not limited to, ammonium lauryl sulfate or sodium lauryl sulfate (SDS). Suitable alkyl ether sulfates include, but are not limited to, sodium laureth sulfate, also known as sodium lauryl ether sulfate (SLES) or sodium myreth sulfate.

Suitable sulfonates include, but are not limited to, docusate (dioctyl sodium sulfosuccinate), fluorosurfactants that are sulfonated and alkyl benzene sulfonates.

Typical sulfonated fluorosurfactants include, but are not limited to, perfluorooctanesulfonate (PFOS) or perfluorobutanesulfonate.

Phosphates are typically alkyl aryl ether phosphates or alkyl ether phosphates.

Carboxylates are typically alkyl carboxylates, such as fatty acid salts (soaps), such as for example, sodium stearate. Alternatively, the carboxylate can be, but is not limited to, sodium lauryl sarcosinate. In another alternative aspect, the carboxylate includes but is not limited to a carboxylated fluorosurfactant, such as perfluorononanoate, or perfluorooctanoate (PFOA or PFO).

When a single surfactant molecule exhibits both anionic and cationic dissociations it is called amphoteric or zwitterionic. Zwitterionic (amphoteric) surfactant is based on primary, secondary or tertiary amines or quaternary ammonium cation also having a sulfonate, carboxylate or a phosphate.

Suitable zwitterionic surfactants include, but are not limited to, CHAPS (3-[(3-Cholamidopropyl)dimethylammonio]-1-propanesulfonate) or a sultaine. The sultaine is typically cocamidopropyl hydroxysultaine.

In one aspect, the carboxylate cation is an amino acid, imino acid or betaine. In one aspect, the betaine is typically cocamidopropyl betaine.

When the zwitterionic surfactant includes a phosphate, lecithin is often chosen as the counterion.

The term “sodium dodecyl sulfate,” “SDS,” “NaDS,” “sodium lauryl sulfate,” or “SLS” refers to an organic compound with the formula CH₃(CH₂)₁₁OSO₃Na), having the CAS Reg. No. 151-21-3.

The term “solubilizer” is intended to include surfactants but more specifically low molar mass surfactants such as propylene glycol (propane-1, 2-diol). Low molar mass surfactants are those having a molar mass of a range of from about 80 to about 300 g/mol, for example an agent having a molar mass of 250 g/mol or less, preferably with a molar mass of 200 g/mol or less, a molar mass of 150 g/mol or less, a molar mass of 100 g/mol or less and/or a molar mass of 80 g/mol or less and mixtures thereof. These low molar mass surfactants include one or more hydroxyl groups, preferably two hydroxyl groups or more.

The term “cleaner” is intended to include surfactants but more specifically low molar mass surfactants that include an ethoxy (ether) portion such as 2-(2-ethoxyethoxy)ethanol (propylene glycol). Low molar mass ethoxy surfactants are those having a molar mass of a range of from about 75 to about 300 g/mol, for example an agent having a molar mass of 250 g/mol or less, preferably with a molar mass of 200 g/mol or less, a molar mass of 150 g/mol or less, a molar mass of 140 g/mol or less, a molar mass of 100 g/mol or less and/or a molar of 80 g/mol or less and mixtures thereof. These low molar mass surfactants include one or more ether (ethoxy) groups, preferably two ethoxy groups or more.

The term “disinfectant” refers to a substance that when applied to non-living objects, destroys microorganisms that are living on the objects. The term “disinfect” refers to the process of destruction or prevention of biological contaminants. Disinfection does not necessarily kill all microorganisms, especially nonresistant bacterial spores; it is less effective than sterilization, which is an extreme physical and/or chemical process that kills all types of life.

Disinfectants are different from other antimicrobial agents such as antibiotics, which destroy microorganisms within the body, and antiseptics, which destroy microorganisms on living tissue. Disinfectants are also different from biocides. The latter are intended to destroy all forms of life, not just microorganisms. Sanitizers are substances that simultaneously clean and disinfect.

The term “sterilant” (via sterilization) refers to a substance that when applied to non-living objects, destroys all viable forms of microbial life, when used according to labeling.

The term “CFU” refers colony forming units and is a measure of viable cells in which a colony represents an aggregate of cells derived from a single progenitor cell.

In various embodiments, the liquid sterilant includes: (a) hydrogen peroxide; (b) an organic acid; (c) a chelator that is not Dequest® 2010 (1-hydroxyethylidene-1,1,-diphosphonic acid), in particular a sulfonic acid containing polymer, copolymer or a support functionalized with sulfonic acid groups; and (d) surfactant.

It should be understood that certain embodiments disclosed herein do not include 1-hydroxyethylidene-1,1,-diphosphonic acid. In embodiments disclosed herein, the liquid sterilants and methods do not leave a residue on a treated surface after use of the liquid sterilant to treat the surface.

It is appreciated that those of ordinary skill in the art fully understand and appreciate that when a liquid sterilant includes more than one component, the liquid sterilant may also include additional components formed as a product of the reaction between the components in the liquid sterilant. For example, those of skill in the art fully understand and appreciate that a liquid sterilant including hydrogen peroxide (H₂O₂) and acetic acid (CH₃CO₂H) also includes the oxidized product of acetic acid, peracetic acid (CH₃CO₃H). As such, reference to the liquid sterilant including hydrogen peroxide (H₂O₂) and acetic acid (CH₃CO₂H) is proper, as well as reference to the liquid sterilant being formed from hydrogen peroxide (H₂O₂) and acetic acid (CH₃CO₂H). To that end, a liquid sterilant of acetic acid and hydrogen peroxide will include significant and appreciable amounts of peracetic acid formed from the reaction of acetic acid with hydrogen peroxide. Further, it is appreciated that those of ordinary skill in the art fully understand and appreciate that an equilibrium exists between hydrogen peroxide and acetic acid, and peracetic acid.

In various embodiments, peracetic acid is present in about 1 wt. % to about 15 wt. % of the liquid sterilant. In some embodiments, peracetic acid is present in about 2-14 wt. %, 3-12 wt. %, 4-11 wt. %, 5-9 wt. %, about 6-8 wt. %, or about 1 wt. %, 2 wt. %, 3 wt. %, 4 wt. %, 5 wt. %, 6 wt. %, 7 wt. %, 8 wt. %, 9 wt. %, 10 wt. %, 11 wt. %, 12 wt. %, 13 wt. %, 14 wt. %, or about 15 wt. % or more of the liquid sterilant. In some embodiments, peracetic acid is present in about 8 wt. % to about 13 wt. % of the liquid sterilant.

In various embodiments, hydrogen peroxide is present in about 10 wt. % to about 50 wt. % of the liquid sterilant. In some embodiments (e.g., before equilibration and formation of PAA), the hydrogen peroxide is present in about 15-45 wt. %, 20-35 wt. %, or about 25-30 wt. % of the liquid sterilant. In some embodiments (e.g., after equilibration and formation of PAA), the hydrogen peroxide is present in about 10-40 wt. %, 15-35 wt. %, 18-30 wt. % or about 20-26 wt. % of the liquid sterilant. In some embodiments, the hydrogen peroxide is present in about 16 wt. %, 18 wt. %, 20 wt. %, 21 wt. %, 22 wt. %, 23 wt. %, 24 wt. %, 25 wt. %, 26 wt. %, 27 wt. %, 28 wt. %, 29 wt. %, 30 wt. %, 31 wt. %, 32 wt. %, 34 wt. %, or about 36 wt. %. In some embodiments, the hydrogen peroxide is about 35 wt. % in water, present in about 18 wt. % to about 32 wt. % of the liquid sterilant. In some embodiments, hydrogen peroxide is about 35 wt. % in water, present in about 28 wt. % of the liquid sterilant. In some embodiments, hydrogen peroxide is about 35 wt. % in water, present in about 17 wt. % to about 26 wt. % of the liquid sterilant.

In various embodiments, the organic acid includes acetic acid. In some embodiments, the organic acid comprises glacial acetic acid. In some embodiments, the organic acid includes acetic acid, present in at least about 3 wt. % of the liquid sterilant. In some embodiments (e.g., before equilibration and formation of PAA), the organic acid includes acetic acid, present in about 1-50 wt. %, 2-45 wt. %, 3-40 wt. %, 4-35 wt. %, 6-30 wt. %, 8-24 wt. %, 10-22 wt. %, 12-20 wt. %, about 14-18 wt. %, or about 4 wt. %, 5 wt. %, 6 wt. %, 7 wt. %, 8 wt. %, 9 wt. %, 10 wt. %, 11 wt. %, 12 wt. %, 13 wt. %, 14 wt. %, 15 wt. %, 16 wt. %, 17 wt. %, 18 wt. %, 19 wt. %, 20 wt. %, 21 wt. %, 22 wt. %, 23 wt. %, 24 wt. %, or about 25 wt. % of the liquid sterilant. In some embodiments (e.g., after equilibration and formation of PAA), the organic acid includes acetic acid, present in about 1-20 wt. %, 2-18 wt. %, 3-17 wt. %, 4-16 wt. %, 5-15 wt. %, 6-14 wt. %, 7-13 wt. %, 8-12 wt. %, or about 9-11 wt. % of the liquid sterilant. In some embodiments, the organic acid includes acetic acid, present in about 13 wt. % to about 17 wt. % of the liquid sterilant. In some embodiments, the organic acid comprises acetic acid, present in about 16 wt. % of the liquid sterilant.

In specific embodiments, the liquid sterilant can be formulated as, can exist as, and can be commercially available as a liquid concentrate disinfectant or sterilant. The term “liquid concentrate” refers to a composition that is relatively undiluted and concentrated, having a low content of carrier, e.g., water. Having the composition be commercially available as a liquid concentrate will typically save costs associated with the manufacturing, shipping, and/or storage of the product.

When the liquid sterilant is formulated as a liquid concentrate, the concentrate can subsequently be diluted with an appropriate amount of carrier (e.g., water) prior to use. Additionally, although considered to be a concentrate, when the liquid sterilant is formulated as a liquid concentrate, a discrete and finite amount of carrier (e.g., water) can be employed.

The liquid sterilant can be formulated for application, depending upon the user's preference as well as the ultimate application of the liquid sterilant. For example, the liquid sterilant can be formulated for use in a sprayable composition, atomized liquid sprayer, or liquid applicator. Such formulations can include at least one of a spray bottle, motorized sprayer, wipe, cloth, sponge, non-woven fabric, and woven fabric. Such formulations may be particularly suitable for applying the liquid sterilant to a surface of a hospital, physician's office, medical clinic, medical facility, dental office, dental facility, airport, school, pet store, zoo, children's day care, elderly nursing home, museum, movie theatre, athletic facility, sporting arena, gymnasium, rest room, bathroom, shopping center, amusement park, church, synagogue, mosque, temple, restaurant, food processing facility, food manufacturing facility, pharmaceutical company, hot-tub, sauna, and/or clean room.

Such liquid formulations may be particularly suitable for applying the liquid sterilant to metal, plastic, natural rubber, synthetic rubber, glass, stone, grout, fiberglass, wood, concrete, construction products, and/or building products.

In various embodiments, the liquid sterilant can be configured for use in contacting at least one of medical equipment, medical device (e.g., reusable medical device or instrument, such as an endoscope), surface in the medical industry, dental equipment, dental device, and surface in the dental industry. In some embodiments, the liquid sterilant may be used in the reconditioning of a soiled endoscopic device. In some embodiments, the liquid sterilants are useful during the disinfection step or sterilization step of the high level disinfection cleaning process following use of the endoscope in a medical procedure. The term “endoscopic device” includes a plurality of minimally invasive surgical devices (e.g., scopes) that have been developed for specific uses. For example, upper and lower endoscopes are utilized for accessing the esophagus/stomach and the colon, respectively, angioscopes are utilized for examining blood vessels, and laparoscopes are utilized for examining the peritoneal cavity.

In some embodiments, catalysts for the formation of peracetic acid from hydrogen peroxide and acetic acid are employed. Suitable catalysts include, for example, inorganic acids, such as sulfuric acid (H₂SO₄), hydrochloric acid (HCl), phosphoric acid (H₃PO₄), and nitric acid (HNO₃).

In specific embodiments, the liquid sterilant can be non-corrosive. The term “non-corrosive” or “noncorrosive” refers to a substance that will not destroy or irreversibly damage another surface or substance with which it comes into contact. The main hazards to people include damage to the eyes, the skin, and the tissue under the skin; inhalation or ingestion of a corrosive substance can damage the respiratory and gastrointestinal tracts. Exposure results in chemical burn. Having the liquid sterilant be relatively non-corrosive will allow the user to employ the liquid sterilant over a wider range of uses, exposing the liquid sterilant to a wider range of substrates. For example, having the liquid sterilant be relatively non-corrosive will allow the user to employ the liquid sterilant as a disinfectant or sterilant with certain medical devices that are highly sensitive to corrosive substances.

In specific embodiments, the liquid sterilant can be non-toxic. The term “non-toxic” refers to a substance that has a relatively low degree to which it can damage a living or non-living organism. Toxicity can refer to the effect on a whole organism, such as an animal, bacterium, or plant, as well as the effect on a substructure of the organism, such as a cell (cytotoxicity) or an organ (organotoxicity), such as the liver (hepatotoxicity). A central concept of toxicology is that effects are dose-dependent; even water can lead to water intoxication when taken in large enough doses, whereas for even a very toxic substance such as snake venom there is a dose below which there is no detectable toxic effect. Having the liquid sterilant be relatively non-toxic will allow a wider range of users be able to safely handle the liquid sterilant, without serious safety concerns or risks.

In specific embodiments, the liquid sterilant can be stable over extended periods of time (i.e., has a long-term stability). The term “long-term stability” refers to a substance undergoing little or no physical and/or chemical decomposition or degradation, over extended periods of time.

In further specific embodiments, the liquid sterilant components (A) and (B) can be stable over extended periods of time, such that at about 1 atm and about 19° C., less than about 20 wt. %, e.g., 15 wt. %, 10 wt. %, or 5 wt. %, of each component independently degrades over about one year. In additional specific embodiments, the liquid sterilant components (A) and (B), as described herein, can be stable over extended periods of time, such that at about 1 atm and about 19° C., at least about 80 wt. % of each component, e.g., 85 wt. %, 90 wt. %, 95 wt. %, is independently present after about one year.

Having the liquid sterilant components be relatively stable over extended periods of time will allow the liquid sterilant to retain its effectiveness over that time, ensuring that it will remain useful and active for its intended purpose. In contrast, in those liquid sterilants that do not retain their effectiveness over that time, product loss can result, which can be financially costly. Additionally, risks associated with the use of a product that has lost some or all of its effectiveness for the intended purpose can be hazardous, in that the product may not effectively achieve the desired goal. For example, when used to disinfect or sterilize a medical device, use of a liquid sterilant that has lost some or all of its effectiveness as a disinfectant or sterilant may not effectively disinfect or sterilize the medical device. Medical injuries can be sustained by the patient, including serious infections.

In specific embodiments, the liquid sterilant of the present includes a buffer. The term “buffer,” “buffering agent,” or “buffering substance” refers to a weak acid or base used to maintain the acidity (pH) of a solution at a chosen value. The function of a buffering agent is to prevent a rapid change in pH when acids or bases are added to the solution. Buffering agents have variable properties—some are more soluble than others; some are acidic while others are basic. A suitable buffering mixture includes a metal hydroxide, such as sodium hydroxide and a metal phosphate dibasic or tribasic, such as potassium phosphate dibasic, e.g., NaOH/K₂HPO₄, K₃HPO₄, NaOH and Na₃PO₄, KOH with a phosphate, etc. The buffer (or its components) can be present in the liquid sterilant in an amount of 4% to about 21% based on the total weight percent of the liquid sterilant.

In specific embodiments, the liquid sterilant can be essentially free of transition metals. In further specific embodiments, the liquid sterilant can include less than about 0.001 wt. % transition metals. In further specific embodiments, the liquid sterilant can include less than about 0.0001 wt. % transition metals. In further specific embodiments, the liquid sterilant can include less than about 0.00001 wt. % transition metals. Having the liquid sterilant include a minimal amount of transition metals decreases the likelihood that the transition metals will cause degradation and/or decomposition of the components (A) and (B) of the liquid sterilant, over the extended periods of time associates with the manufacturing, shipping, and storage of the components.

The term “transition metal,” “transition metals” or “transition element” refers to an element whose atom has an incomplete d sub-shell, or which can give rise to cations with an incomplete d sub-shell. Transition metals include scandium (Sc), titanium (Ti), vanadium (V), chromium (Cr), manganese (Mn), iron (Fe), cobalt (Co), nickel (Ni), copper (Cu), zinc (Zn), yttrium (Y), zirconium (Zr), niobium (Nb), molybdenum (Mo), technetium (Tc), ruthenium (Ru), rhodium (Rh), palladium (Pd), silver (Ag), cadmium (Cd), hafnium (Hf), tantalum (Ta), tungsten (W), rhenium (Re), osmium (Os), iridium (Ir), platinum (Pt), gold (Au), mercury (Hg), rutherfordium (Rf), dubnium (Db), seaborgium (Sg), bohrium (Bh), hassium (Hs) and copernicium (Cn).

In specific embodiments, the transition metal can be naturally occurring. Naturally occurring transition metals include scandium (Sc), titanium (Ti), vanadium (V), chromium (Cr), manganese (Mn), iron (Fe), cobalt (Co), nickel (Ni), copper (Cu), zinc (Zn), yttrium (Y), zirconium (Zr), niobium (Nb), molybdenum (Mo), technetium (Tc), ruthenium (Ru), rhodium (Rh), palladium (Pd), silver (Ag), cadmium (Cd), hafnium (Hf), tantalum (Ta), tungsten (W), rhenium (Re), osmium (Os), iridium (Ir), platinum (Pt), gold (Au), and mercury (Hg).

In specific embodiments, the liquid sterilant can be essentially free of heavy metals. In further specific embodiments, the liquid sterilant can include less than about 0.001 wt. % heavy metals. In further specific embodiments, the liquid sterilant can include less than about 0.0001 wt. % heavy metals. In further specific embodiments, the liquid sterilant can include less than about 0.00001 wt. % heavy metals. Having the liquid sterilant include a minimal amount of heavy metals decreases the likelihood that the transition metals will cause degradation and/or decomposition of components (A) and (B) of the liquid sterilant, over the extended periods of time associates with the manufacturing, shipping, and storage of the composition.

The term “heavy metal,” “heavy metals” or “toxic metal” refers to metals that are relatively toxic, and mainly include the transition metals, some metalloids, lanthanides, and actinides. Examples of toxic metals include, e.g., iron (Fe), cobalt (Co), copper (Cu), manganese (Mn), molybdenum (Mo), zinc (Zn), mercury (Hg), plutonium (Pu), lead (Pb), vanadium (V), tungsten (W), cadmium (Cd), aluminium (Al), beryllium (Be), and arsenic (As).

The present embodiments also provide for kits that include: (a) enclosed containers that include removable closures; (b) the components (A) and (B) of the liquid sterilant as described herein, which are located inside the enclosed containers; and (c) printed indicia located on the enclosed containers.

In specific embodiments, the enclosed container can be opaque. In additional specific embodiments, the enclosed container can be manufactured from high density polyethylene (HDPE), thereby providing the requisite opacity. Having the enclosed container be manufactured from high density polyethylene (HDPE) will decrease the likelihood that components (A) and (B) will degrade and/or decompose over extended periods of time, due to excessive exposure to direct sunlight.

The term “high-density polyethylene” or “HDPE” refers to a polyethylene thermoplastic made from petroleum. The mass density of high-density polyethylene can range from 0.93 to 0.97 g/cm³. Although the density of HDPE is only marginally higher than that of low-density polyethylene, HDPE has little branching, giving it stronger intermolecular forces and tensile strength than LDPE. The difference in strength exceeds the difference in density, giving HDPE a higher specific strength. It is also harder and more opaque and can withstand somewhat higher temperatures (120° C./248° F. for short periods, 110° C./230° F. continuously). HDPE is resistant to many different solvents.

The term “solvent” as used herein refers to a liquid that can dissolve a solid, liquid, or gas. Non-limiting examples of solvents are silicones, organic compounds, water, alcohols, ionic liquids, and supercritical fluids.

The term “opaque” refers to an object that is neither transparent (allowing all light to pass through) nor translucent (allowing some light to pass through). When light strikes an interface between two substances, in general some may be reflected, some absorbed, some scattered, and the rest transmitted (also see refraction). Reflection can be diffuse, for example light reflecting off a white wall, or specular, for example light reflecting off a mirror. An opaque substance transmits no light, and therefore reflects, scatters, or absorbs all of it. Both mirrors and carbon black are opaque. Opacity depends on the frequency of the light being considered. For instance, some kinds of glass, while transparent in the visual range, are largely opaque to ultraviolet light. More extreme frequency-dependence is visible in the absorption lines of cold gases.

To further decrease the likelihood that components (A) and (B) will degrade and/or decompose over extended periods of time, components (A) and (B) of the liquid sterilant should avoid, when feasible: excessive exposure to direct sunlight, excessive heat and/or elevated temperatures. As such, in specific embodiments, the enclosed container of the kit can include printed indicia, with instructions to avoid excessive heat, elevated temperatures, direct sunlight, or a combination thereof.

Over extended periods of time, hydrogen peroxide and/or peracetic acid present in the liquid sterilant will be susceptible to degrade or decompose (and a portion of the hydrogen peroxide may degrade or decompose), thereby evolving oxygen.

In specific embodiments, the enclosed containers includes a head space, pressure valve, or combination thereof. In specific embodiments, the enclosed container includes a pressure valve, configured to release excessive gas from within the enclosed container. The presence of a head space and pressure valve in the container will allow for the escape of gas (e.g., oxygen) from the enclosed container, without the likelihood that the container will explode from the elevated pressure that would otherwise develop.

The term “head space” refers to a portion of the inside of a container that is not occupied by the liquid contents of the container. In particular, when a container includes a liquid composition, a head space can be present in the container such that a portion of the inside of the container does not include liquid composition, but instead includes a gas or vacuum. In specific embodiments, the head space can include oxygen (O₂), peracetic acid and/or acetic acid vapor. In further specific embodiments, the head space can be present in up to about 20% (v/v) of the inside of the enclosed container.

The term “pressure valve” refers to a mechanical device that will permit for the passage of gas and not fluid, preferably in one direction only, for example, exiting a container housing the pressure valve, and not entering the container.

The liquid sterilant can be used to effectively reduce the number of microbes located upon a substrate. In specific embodiments, the liquid sterilant can effectively kill and/or inhibit a microorganism (e.g., virus, fungus, mold, slime mold, algae, yeast, mushroom and/or bacterium), thereby disinfecting or sterilizing the substrate.

In additional specific embodiments, the liquid sterilant can effectively sanitize a substrate, thereby simultaneously cleaning and disinfecting and/or sterilizing the substrate. In additional specific embodiments, the liquid sterilant can effectively kill or inhibit all forms of life, not just microorganisms, thereby acting as a biocide.

In specific embodiments, the liquid sterilant can effectively disinfect or sterilize a substrate. In further specific embodiments, the liquid sterilant can effectively disinfect or sterilize the surface of a substrate. In additional specific embodiments, the liquid sterilant can effectively sterilize a substrate. In further specific embodiments, the liquid sterilant can effectively sterilize the surface of a substrate.

The term “microbe,” “microbes” “microorganism,” or “micro-organism” refers to a microscopic organism that comprises either a single cell (unicellular), cell clusters, or no cell at all (acellular). Microorganisms are very diverse; they include bacteria, fungi, archaea, and protists; microscopic plants (green algae); and animals such as plankton and the planarian. Some microbiologists also include viruses, but others consider these as non-living. Most microorganisms are unicellular (single-celled), but this is not universal, since some multicellular organisms are microscopic, while some unicellular protists and bacteria, like Thiomargarita namibiensis, are macroscopic and visible to the naked eye.

The term “virus” refers to a small infectious agent that can replicate only inside the living cells of organisms. Virus particles (known as virions) consist of two or three parts: the genetic material made from either DNA or RNA, long molecules that carry genetic information; a protein coat that protects these genes; and in some cases an envelope of lipids that surrounds the protein coat when they are outside a cell. The shapes of viruses range from simple helical and icosahedral forms to more complex structures. The average virus is about one one-hundredth the size of the average bacterium. An enormous variety of genomic structures can be seen among viral species; as a group they contain more structural genomic diversity than plants, animals, archaea, or bacteria. There are millions of different types of viruses, although only about 5,000 of them have been described in detail. A virus has either DNA or RNA genes and is called a DNA virus or a RNA virus respectively. The vast majority of viruses have RNA genomes. Plant viruses tend to have single-stranded RNA genomes and bacteriophages tend to have double-stranded DNA genomes.

The term “fungi” or “fungus” refers to a large and diverse group of eucaryotic microorganisms whose cells contain a nucleus, vacuoles, and mitochondria. Fungi include algae, molds, yeasts, mushrooms, and slime molds. See, Biology of Microorganisms, T. Brock and M. Madigan, 6th Ed., 1991, Prentice Hill (Englewood Cliffs, N.J.). Exemplary fungi include Ascomycetes (e.g., Neurospora, Saccharomyces, Morchella), Basidiomycetes (e.g., Amanita, Agaricus), Zygomycetes (e.g., Mucor, Rhizopus), Oomycetes (e.g., Allomyces), and Deuteromycetes (e.g., Penicillium, Aspergillus).

The term “mold” refers to a filamentous fungus, generally a circular colony that may be cottony, wooly, etc. or glabrous, but with filaments not organized into large fruiting bodies, such as mushrooms. See, e.g., Stedman's Medical Dictionary, 25th Ed., Williams & Wilkins, 1990 (Baltimore, Md.). One exemplary mold is the Basidiomycetes called wood-rotting fungi. Two types of wood-rotting fungi are the white rot and the brown rot. An ecological activity of many fungi, especially members of the Basidiomycetes is the decomposition of wood, paper, cloth, and other products derived from natural sources. Basidiomycetes that attack these products are able to utilize cellulose or lignin as carbon and energy sources. Lignin is a complex polymer in which the building blocks are phenolic compounds. It is an important constituent of woody plants. The decomposition of lignin in nature occurs almost exclusively through the agency of these wood-rotting fungi. Brown rot attacks and decomposes the cellulose and the lignin is left unchanged. White rot attacks and decomposes both cellulose and lignin. See, Biology of Microorganisms, T. Brock and M. Madigan, 6th Ed., 1991, Prentice Hill (Englewood Cliffs, N.J.).

The term “slime molds” refers to nonphototrophic eucaryotic microorganisms that have some similarity to both fungi and protozoa. The slime molds can be divided into two groups, the cellular slime molds, whose vegetative forms are composed of single amoeba like cells, and the acellular slime molds, whose vegetative forms are naked masses of protoplasms of indefinite size and shape called plasmodia. Slime molds live primarily on decaying plant matter, such as wood, paper, and cloth. See, Biology of Microorganisms, T. Brock and M. Madigan, 6th Ed., 1991, Prentice Hill (Englewood Cliffs, N.J.).

The term “algae” refers to a large and diverse assemblage of eucaryotic organisms that contain chlorophyll and carry out oxygenic photosynthesis. See, Biology of Microorganisms, T. Brock and M. Madigan, 6th Ed., 1991, Prentice Hill (Englewood Cliffs, N.J.). Exemplary algae include Green Algae (e.g., Chlamydomonas), Euglenids (e.g., Euglena), Golden Brown Algae (e.g., Navicula), Brown Algae (e.g., Laminaria), Dinoflagellates (e.g., Gonyaulax), and Red Algae (e.g., Polisiphonia).

The term “yeast” refers to unicellular fungi, most of which are classified with the Ascomytes. See, Biology of Microorganisms, T. Brock and M. Madigan, 6th Ed., 1991, Prentice Hill (Englewood Cliffs, N.J.).

The term “mushrooms” refer to filamentous fungi that are typically from large structures called fruiting bodies, the edible part of the mushroom. See, Biology of Microorganisms, T. Brock and M. Madigan, 6th Ed., 1991, Prentice Hill (Englewood Cliffs, N.J.).

The term “bacterium” or “bacteria” refers to a large domain of prokaryotic microorganisms. Typically a few micrometers in length, bacteria have a wide range of shapes, ranging from spheres to rods and spirals. Bacteria are present in most habitats on Earth, growing in soil, acidic hot springs, radioactive waste, water, and deep in the Earth's crust, as well as in organic matter and the live bodies of plants and animals, providing outstanding examples of mutualism in the digestive tracts of humans, termites and cockroaches. There are typically about 40 million bacterial cells in a gram of soil and a million bacterial cells in a milliliter of fresh water; in all, there are approximately five nonillion (5×10³⁰) bacteria on Earth, forming a biomass that exceeds that of all plants and animals. Most bacteria have not been characterized, and only about half of the phyla of bacteria have species that can be grown in the laboratory.

The term “P. aeruginosa” or “Pseudomonas aeruginosa” refers to a common bacterium that can cause disease in animals, including humans. It is found in soil, water, skin flora, and most man-made environments throughout the world. It thrives not only in normal atmospheres, but also in hypoxic atmospheres, and has, thus, colonized many natural and artificial environments. It uses a wide range of organic material for food; in animals, the versatility enables the organism to infect damaged tissues or those with reduced immunity. The symptoms of such infections are generalized inflammation and sepsis. If such colonizations occur in critical body organs, such as the lungs, the urinary tract, and kidneys, the results can be fatal. Because it thrives on most surfaces, this bacterium is also found on and in medical equipment, including catheters, causing cross-infections in hospitals and clinics. It is implicated in hot-tub rash.

The term “S. aureus” or “Staphylococcus aureus” refers to a facultative anaerobic Gram-positive bacterium. It is frequently found as part of the normal skin flora on the skin and nasal passages. It is estimated that 20% of the human population are long-term carriers of S. aureus. S. aureus is the most common species of staphylococci to cause Staph infections. The reasons S. aureus is a successful pathogen are a combination host and bacterial immuno-evasive strategies. One of these strategies is the production of carotenoid pigment staphyloxanthin which is responsible for the characteristic golden color of S. aureus colonies. This pigment acts as a virulence factor, primarily being a bacterial antioxidant which helps the microbe evade the host's immune system in the form of reactive oxygen species which the host uses to kill pathogens.

S. aureus can cause a range of illnesses from minor skin infections, such as pimples, impetigo, boils (furuncles), cellulitis folliculitis, carbuncles, scalded skin syndrome, and abscesses, to life-threatening diseases such as pneumonia, meningitis, osteomyelitis, endocarditis, toxic shock syndrome (TSS), bacteremia, and sepsis. Its incidence is from skin, soft tissue, respiratory, bone, joint, endovascular to wound infections. It is still one of the five most common causes of nosocomial infections, often causing postsurgical wound infections. Each year, some 500,000 patients in American hospitals contract a staphylococcal infection.

Methicillin-resistant S. aureus, abbreviated MRSA and often pronounced “mer-sa” (in North America), is one of a number of greatly-feared strains of S. aureus which have become resistant to most antibiotics. MRSA strains are most often found associated with institutions such as hospitals, but are becoming increasingly prevalent in community-acquired infections.

The term “E. hirae” or “Enterococcus hirae” refers to a species of Enterococcus.

The term “M. terrae” or “Mycobacterium terrae” refers to a slow-growing species of Mycobacterium. It is an ungrouped member of the third Runyon (nonchromatogenic mycobacteria). It is known to cause serious skin infections, which are relatively resistant to antibiotic therapy

The term “Mycobacterium avium complex,” “M. avium complex” or “MAC” refers to a group of genetically related bacteria belonging to the genus Mycobacterium. It includes Mycobacterium avium and Mycobacterium intracellulare.

The term “M. avium” or “Mycobacterium avium” refers to a species of Mycobacterium.

The term “M. intracellulare” or “mycobacterium intracellulare” refers to a species of Mycobacterium.

The following paragraphs enumerated consecutively from 1 through 32 provide for various aspects of the present invention.

In one embodiment, in a first paragraph (1), the present invention provides a two part liquid sterilant composition comprising components (A) and (B) to be mixed to yield an aqueous composition, wherein component (A) comprises hydrogen peroxide, acetic acid and/or peracetic acid and a stabilizer; and component (B) comprises a buffer, an anticorrosive agent, a solubilizer and a cleaner, wherein when component (A) and component (B) are combined, form a liquid sterilant.
2. The two part liquid composition of paragraph 1, wherein the stabilizer is a phosphonic based chelator or a polymeric sulfonic acid resin based chelator.
3. The two part liquid composition of paragraph 2, wherein the phosphonic based chelator comprises 1-hydroxyethylidene-1,1,-diphosphonic acid.
4. The two part liquid composition of paragraph 3, wherein the phosphonic based chelator is present in a range of from about 0.1% to about 1% based on the total weight percent of the liquid sterilant.
5. The two part liquid composition of paragraph 2, wherein the polymeric sulfonic resin functionalized resin is a divinyl benzene/styrene copolymer, a perfluorosulfonic acid resin or a polymer containing a 2-acrylamido-2-methylpropane sulfonic acid resin.
6. The two part liquid composition of paragraph 5, wherein the polymeric sulfonic resin functionalized resin chelator is present in a range of from about 0.25% to about 1% based on the total weight percent of the liquid sterilant.
7. The two part liquid composition of any of paragraphs 1 through 6, wherein the buffer comprises sodium hydroxide and potassium phosphate dibasic.
8. The two part liquid composition of paragraph 7, wherein the sodium hydroxide is present in a range of from about 3% to about 4.5% based on the total weight percent of the liquid sterilant.
9. The two part liquid composition of paragraph 7, wherein the potassium phosphate dibasic is present in a range of from about 14% to about 18% based on the total weight percent of the liquid sterilant.
10. The two part liquid composition of any of paragraphs 1 through 9, wherein the anticorrosive is benzotriazole.
11. The two part liquid composition of paragraph 10, wherein the benzotriazole is present in a range of from about 0.25% to about 1% based on the total weight percent of the liquid sterilant.
12. The two part liquid composition of any of paragraphs 1 through 11, wherein the solubilizer is propylene glycol.
13. The two part liquid composition paragraph 12, wherein the propylene glycol is present in a range of from about 9.5% to about 10.5% based on the total weight percent of the liquid sterilant.
14. The two part liquid composition of any of paragraphs 1 through 13, wherein the cleaner is diethylene glycol mono-ethyl ether.
15. The two part liquid composition of paragraph 14, wherein the diethylene glycol mono-ethyl ether is present in a range of from about 4.75% to about 5.25% based on the total weight percent of the liquid sterilant.
16. The two part liquid composition of paragraph 1, wherein the stabilizer is present in a range of from about 0.1% to about 1%, the buffer is present in a range of from about 3% to about 21%, the anticorrosive is present in a range of from about 0.25% to about 1%, the solubilizer is present in a range of from about 9.5% to about 10.5% and the cleaner is present in a range of from about 4.75% to about 5.25% based on the total weight percent of the liquid sterilant.
17. The two part liquid composition of paragraph 1, wherein the stabilizer is present in a range of from about 0.1% to about 1%, sodium hydroxide is present in a range of from about 3.35% to about 4.5%, potassium phosphate dibasic is present in a range of from about 14.2% to about 18%, benzotriazole is present in a range of from about 0.25% to about 1%, propylene glycol is present in a range of from about 9.5% to about 10.5% and diethylene glycol mono-ethyl ether is present in a range of from about 4.75% to about 5.25% based on the total weight percent of the liquid sterilant.
18. The two part liquid composition of paragraph 17, wherein the stabilizer is present at 0.1%, the sodium hydroxide is present at 3.35%, the potassium phosphate dibasic is present at 18%, the benzotriazole is present at 1%, the propylene glycol is present 10% and the diethylene glycol mono-ethyl ether is present 5% based on the total weight percent of the liquid sterilant.
19. The two part liquid composition of any of paragraphs 1 through 18, wherein the composition does not include sulfuric acid.
20. The two part liquid composition of any of paragraphs 1 through 18, wherein the composition does not include EDTA.
21. The two part liquid composition of any of paragraphs 1 through 20, wherein the component (A), component (B) and water are mixed in a ratio of 0.5:0.5:49 to 5:5:40 to provide the liquid sterilant.
22. The two part liquid composition of any of paragraphs 1 through 21, wherein the liquid sterilant has a peracetic acid concentration of from about 2000 ppm PAA to about 3000 ppm PAA or greater.
23. The two part liquid composition of paragraph 22, wherein the liquid sterilant has a peracetic acid concentration of from about 2000 ppm PAA to about 3000 ppm PAA.
24. The two part liquid composition of any of paragraphs 1 through 23, wherein the liquid sterilant has a pH of greater than 5 to and less than 6.5.
25. The two part liquid composition of paragraph 24, wherein the liquid sterilant has a pH of 5 to 5.5.
26. The two part liquid composition of any of paragraphs 1 through 25, wherein the liquid sterilant maintains a concentration above the minimum required concentration for 24 hours.
27. A method to clean and disinfect or sterilize a substrate contaminated with microorganisms using the two part liquid composition according to any of paragraphs 1 through 26, comprising the step:
   contacting the substrate with the liquid sterilant to clean and disinfect or sterilize the substrate.
28. The method of paragraph 27, wherein the contaminated substrate is treated with the liquid sterilant at a temperature of 30° C. to 40° C.
29. The method of either paragraph 27 or 28, wherein the contaminated substrate is contacted with the liquid sterilant for 5 to 15 minutes.
30. The method of any of paragraphs 27 through 29, wherein the contaminated substrate is disinfected.
31. The method of any of paragraphs 27 through 29, wherein the contaminated substrate is sterilized.
32. A kit comprising:
container (A) and container (B), wherein container A contains component (A) and container B contains component (B) as paragraphed in any of paragraphs 1 through 21;
instructions for combining components (A) and (B) to provide the liquid sterilant composition; and
instructions for contacting a substrate contaminated with microorganisms using the liquid sterilant composition, wherein the substrate is disinfected and/or sterilized.

The invention will be further described with reference to the following non-limiting Examples. It will be apparent to those skilled in the art that many changes can be made in the embodiments described without departing from the scope of the present invention. Thus the scope of the present invention should not be limited to the embodiments described in this application, but only by embodiments described by the language of the claims and the equivalents of those embodiments. Unless otherwise indicated, all percentages are by weight.

### EXAMPLES

For the following examples, all of the part A's of the 2 part chemistries were the same. The formulation developed for Part A is shown in Table 1. For all testing, the concentrate chemistries were prepared by making the solution with the defined concentration of each component listed in Tables 1, 3 and 6 below with the respective Part A and Part B of the chemistry. During testing of the actual mixtures (with Part A and Part B diluted with water), the mixtures (also identified as the “Use Solution”) for sterilization were made by diluting Part A, Part B and water with the ratio of 1:1:48 (1 Part A: 1 Part B: and 48 Part water) by mass.

Unless otherwise specified, for the examples below, Part A and Part B were added to water, mixed and used within 8 hours. For the suspension test and AOAC carrier test for microbiological efficacy testing, the chemistry was used within 4-6 hours to ensure that the PAA concentration were within the desired range of 2000-2100 ppm PAA at 30±2° C.

The chemistry in the examples was designed to work at 30-40° C. temperature range and can be used in an automated endoscope reprocess (AER) which mixes the Part A and Part B with water and typically uses it within 5-15 minutes (which is the contact time of the chemistry). Nominal PAA concentrations were achieved using the dilution ratio of 1:1:48 (Part A to Part B to water).

Table 1 shows the formulation for Part A. Note: The information provided in the table below defines the range of chemistry at each stage of the chemistry life.

TABLE 1

| | Hydrogen | | | Stabilizer |
|---|---|---|---|---|
| | Peroxide | Acetic Acid | Peracetic Acid | (Dequest 2020) |
| | Concentration | Concentration | Concentration | Concentration |
| Formulation | wt % | (wt %) | (wt %) | (wt %) |
| Initial mixture | 29.34% to 30.66% | 24.30% to 25.70% | Not Applicable | 0.09% to 0.11% |
| range (brew | | | | |
| concentration | | | | |
| range) | | | | |
| Final specification | 23.4% to 26.9% | 13.5% to 17% | 11.0% to 13.0% | 0.09% to 0.11% |
| range | | | | |
| (Equilibrium after | | | | |
| production | | | | |
| process) | | | | |
| Estimated range | 15.5% to 26.9% | 13.5% to 17% | 8.25% to 13% | 0.09% to 0.11% |
| for lifetime of | | | | |
| chemistry | | | | |

Stability Study of Part A of 2 part Chemistry—For Part A chemistry, a stability study was done to show the stability of the chemistry over time. For the Part A of the chemistry the main criteria for the chemistry stability was for the PAA concentration to not drop below 9.3% PAA over time. For hydrogen peroxide, a stability of hydrogen peroxide greater than 20% was desired to ensure the stability of the chemistry over time. For acetic acid, a stability greater than 13% over time was desired.

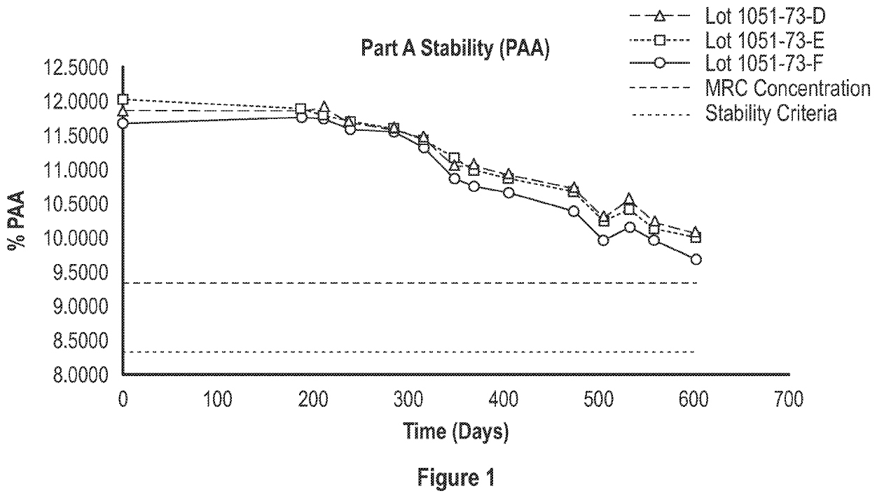

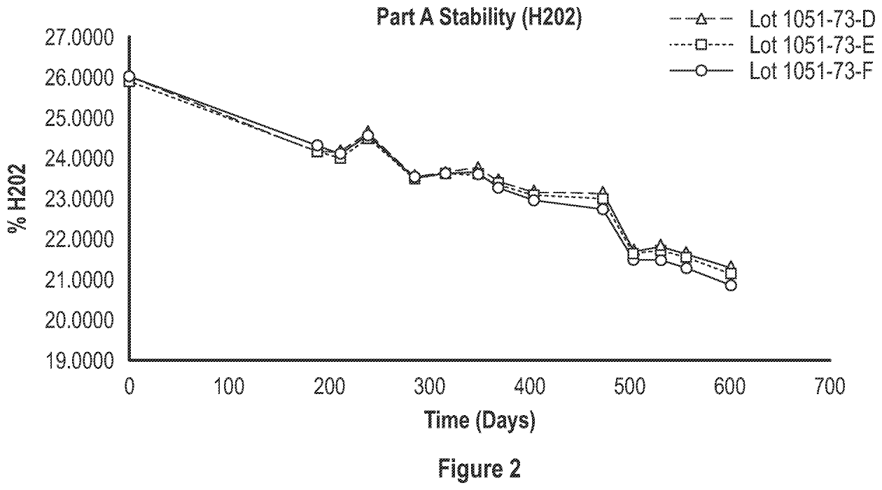

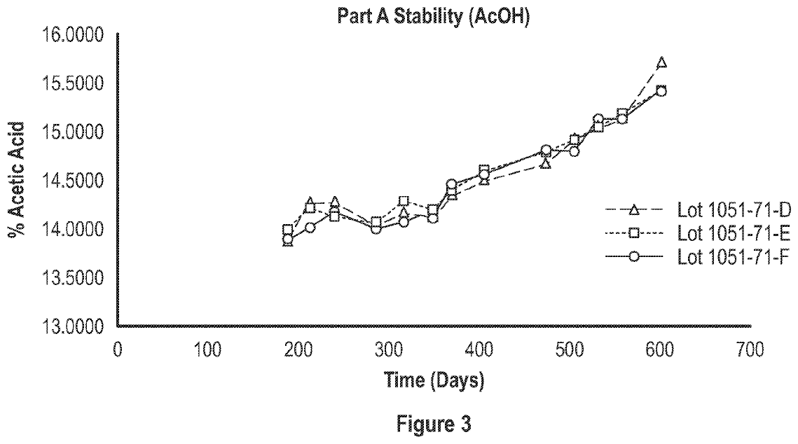

For this study, three different lots of Part A having the formulation in Table 1 were made and kept for monitoring over time. FIGS. 1 to 3 show the stability of the three different lots. FIG. 1 shows the PAA stability data for the stability study of Part A. The data showed that after 653 days at ambient conditions, the PAA concentration of all three lots were still greater than the criteria of 9.3% PAA. FIG. 2 shows the hydrogen peroxide stability data for the stability study of Part A. The results showed that after 653 days at ambient conditions, the chemistry still had greater than 20% hydrogen peroxide for all three lots. FIG. 3 shows the acetic acid stability data for the stability study of Part A. The results showed that over the lifetime of the stability study at ambient conditions, the acetic acid maintained the concentration above 13%.

Part B formulations and development detail: For Part B formulations, Table 2 shows all the definitions of the abbreviations used in the formulation list in Tables 3 and 4.

Table 2 shows the abbreviations used for chemical components listed in the Formulation List for Part B in Table 3 and 6.

TABLE 2

| | | Chemical |
|---|---|---|
| Abbreviation | Component Name | Characteristic |
| NaOH | Sodium Hydroxide | Buffer component |
| K₂HPO₄ | Potassium Phosphate Dibasic | Buffer component |
| K₃PO₄ | Potassium Phosphate Tribasic | Buffer component |
| Na₃PO₄ | Sodium Phosphate Tribasic | Buffer component |
| C-99 | Benzotriazole (also known as Cobratec | Anticorrosive |
| | 99) | component |
| EDTA | Ethylene Diamine tetra-acetic | Chelator |
| PG | Propylene glycol | Solubilizer |
| DGME | Diethylene Glycol Mono-ethyl Ether | Cleaner |

Corrosion Test Procedure—For the corrosion test study, ASTM G31-72: Standard Practice for Laboratory Immersion Corrosion Testing of Metals was used. For the testing done in the chemistry development process, the method was simplified to the following procedure for faster test time while still allowing sufficient sensitivity in the data for comparison of different formulation: ASTM Method: G31-72 (Reapproved 2004): Standard Practice for Laboratory Immersion Corrosion Testing of Metals was followed utilizing stainless steel (SS316) 316 and brass coupons. The metal coupons were cleaned with acetone and allowed to dry. The coupons were weighed and placed in test tubes. The solutions to be tested were placed in the test tubes. Rapicide™ PA available from Medivators, Inc. was used as a comparison. DI water was used as a control. The coupons were soaked in the test tube for 5 hours at room temperature. After soaking, the solutions were poured out and the coupons were rinsed with DI water. The brass coupons were sonicated in 10% v/v of sulfuric acid for 2 minutes, then rinsed with DI water again. The coupons were then allowed to air dry for 24 hours. Using a 1 kg weigh, the coupons were rubbed with a scrubber utilizing a horizontal force 10 times back and forth on both sides. The coupons were then cleaned with acetone and allowed to dry. The coupons were then reweighed to determine the weight loss per minute for corrosion rate (unit in mg/min or μg/min depending on the need of the experiment).

Suspension Test Procedure—Prior to testing, the germicide was equilibrated at 30° C. by placing the respective bottles in a water bath. The temperature of the germicide was measured by placing a bottle of DI water with the same volume as the germicide into the water bath at the same time as the germicide. A thermometer was placed into the DI water to check for the moment when the temperature stabilized. 1.0 ml of inoculum and 0.5 mL of fetal bovine serum (FBS) was pipetted into a test tube and immediately placed in the water bath at 30° C. for 2 minutes±10 seconds. 8.5 mL of germicide was then added into the test tube (Tube A) containing the inoculum and the FBS, the timer was restarted, and the solution was mixed in the test tube and returned to the water bath. Shortly before the contact time (t), the germicide/inoculum suspension was brought into a biosafety cabinet and vortexed or pipetted up and down to mix the solution. At the contact time (t±10 s) 1.0 mL of the germicide/inoculum suspension was removed and pipetted into 9 mL of appropriate sterile neutralizer (Tube B) for the germicide. For additional time points, the suspension was placed back into the water bath and additional 1.0 mL aliquots were removed from the suspension and pipetted into 9 mL of neutralizer. Appropriate dilutions were then made if needed to obtain countable plates. 1 mL was then transferred from each dilution to two 25 ml saline tubes. The samples were then filtered through 0.20 μm membrane filters the filters were plated in the appropriate agar plates.

AOAC Carrier Test Procedure—The procedure was done following the guideline AOAC Official Method 966.04 but was slightly modified to adapt to the 30° C. contact time with 120 carriers per test for preliminary/feasibility testing.

For Part B development, the formulation summary is divided into two different tables. Table 3 lists the initial Part B formulations. Table 6 shows the Part B formulations with microbiological efficacy testing. For the components listed in Tables 3 and 6, the abbreviation for the component listed can be found in Table 2.

Table 3 shows the formulations for Part B Note: The composition listed are defined in weight percent.

TABLE 3

| Formulation | Buffer | Anticorrosive | Solubilizer | Cleaner | Chelator |
|---|---|---|---|---|---|
| ID | components | component | component | component | component |
| A | 4.46% NaOH | 1% C-99 | 10% PG | 5% DGME | 1% EDTA |
| | and 14.2% | | | | |
| | K₂HPO₄ | | | | |
| B | 4.46% NaOH | 0.5% C-99 | 10% PG | 5% DGME | 1% EDTA |
| | and 14.2% | | | | |
| | K₂HPO₄ | | | | |
| C | 4.5% NaOH and | 0.25% C-99 | 10% PG | 5% DGME | 1% EDTA |
| | 14.2% K₂HPO₄ | | | | |
| D | 4.5% NaOH and | 0.5% C-99 | 10% PG | 5% DGME | 1% EDTA |
| | 14.2% K₂HPO₄ | | | | |
| E | 4.5% NaOH and | 0.75% C-99 | 10% PG | 5% DGME | 1% EDTA |
| | 14.2% K₂HPO₄ | | | | |
| F | 4.5% NaOH and | 1.00% C-99 | 10% PG | 5% DGME | 1% EDTA |
| | 14.2% K₂HPO₄ | | | | |
| G | 4.5% NaOH and | 0.5% C-99 | | | |
| | 14.2% K₂HPO₄ | | | | |
| H | 4.5% NaOH and | 0.5% C-99 | | | 1% EDTA |
| | 14.2% K₂HPO₄ | | | | |
| I | 4.5% NaOH and | 0.5% C-99 | | 5% DGME | 1% EDTA |
| | 14.2% K₂HPO₄ | | | | |
| J | 4.5% NaOH and | 0.5% C-99 | 10% PG | 5% DGME | 1% EDTA |
| | 14.2% K₂HPO₄ | | | | |
| K | 4.46% NaOH | 0.5% C-99 | 10% PG | 5% DGME | 1% EDTA |
| | and 14.2% | | | | |
| | K₂HPO₄ | | | | |
| L | 3.35% NaOH | | | | |
| | and 18% | | | | |
| | K₂HPO₄ | | | | |
| M | 3.35% NaOH | 0.25% C-99 | | | |
| | and 18% | | | | |
| | K₂HPO₄ | | | | |
| N | 3.35% NaOH | 0.50% C-99 | | | |
| | and 18% | | | | |
| | K₂HPO₄ | | | | |
| O | 3.35% NaOH | 0.75% C-99 | | | |
| | and 18% | | | | |
| | K₂HPO₄ | | | | |
| P | 3.35% NaOH | 1.00% C-99 | | | |
| | and 18% | | | | |
| | K₂HPO₄ | | | | |
| Q | 3.35% NaOH | | | | 1% EDTA |
| | and 18% | | | | |
| | K₂HPO₄ | | | | |
| R | 3.35% NaOH | 1.00% C-99 | | | 1% EDTA |
| | and 18% | | | | |
| | K₂HPO₄ | | | | |

Use solutions were made using the various formulations of Part B from Table 3. Table 4 shows the corrosion results.

TABLE 4

| Formulation ID for Part B from Table 3 | Corrosion Rate (mg/min) |
|---|---|
| G | 0.1640 |
| H | 0.0867 |
| I | 0.0233 |
| J | 0.0403 |

The results above indicate that EDTA had a negative impact on the corrosive properties. Both diethylene glycol monoethyl ether and propylene glycol help improve the corrosive properties.

Table 5 shows the effect of different concentration of benzotriazole and the presence of EDTA on corrosive properties. The results of the corrosion tests on Use Solutions utilizing the various Part B formulations from Table 3 are shown in Table 5.

TABLE 5

| Formulation ID for Part B from Table 3 | Corrosion Rate (mg/min) |
|---|---|
| DI Water control | 0.0080 |
| L | 0.0335 |
| M | 0.0223 |
| N | 0.0219 |
| O | 0.0174 |
| P | 0.0161 |
| Q | 0.0348 |
| R | 0.0232 |
| Rapicide PA Double Dose Control | 0.0116 |

The Results show that 0.75% to 1.0% benzotriazole had the best corrosion rate. EDTA was confirmed to increase corrosion rate of the new 2 part chemistry.

Formulations for Part B were then developed based on the corrosion test results for Use Solutions based on the Part B formulations from Table 3 and were generated with microbiological efficacy testing for the formulation selection. Table 6 shows those formulations. Note: The compounds used in each category are listed in the formulation detail with the concentration listed in weight percent.

TABLE 6

| Formulation | Buffer | Anticorrosive | Solubilizer | Cleaner | Chelator |
|---|---|---|---|---|---|
| ID | components | component | component | component | component |
| 1 | 4.5% NaOH and | 0.5% C-99 | | | |
| | 14.2% K₂HPO₄ | | | | |
| 2 | 4.5% NaOH and | 0.5% C-99 | | | 1% EDTA |
| | 14.2% K₂HPO₄ | | | | |
| 3 | 4.5% NaOH and | 0.5% C-99 | | 5% DGME | |
| | 14.2% K₂HPO₄ | | | | |
| 4 | 4.5% NaOH and | 0.5% C-99 | 10% PG | | |
| | 14.2% K₂HPO₄ | | | | |
| 5 | 4.5% NaOH and | 0.5% C-99 | 10% PG | 5% DGME | 1% EDTA |
| | 14.2% K₂HPO₄ | | | | |
| 6 | 4.5% NaOH and | 0.5% C-99 | 10% PG | 5% DGME | 1% EDTA |
| | 24.2% K₂HPO₄ | | | | |
| 8 | 4.3% Na₃PO₄ | 1.00% C-99 | | | |
| 9 | 21.2% K₃PO₄ | 1.00% C-99 | | | |
| 10 | 5.3% K₃PO₄ | 1.00% C-99 | | | |
| 11 | 3.5% NaOH and | 1.00% C-99 | | | |
| | 18% K₂HPO₄ | | | | |
| 12 | 21.2% K₃PO₄ | 1.00% C-99 | 10% PG | 5% DGME | |
| 13 | 3.35% NaOH | 1.00% C-99 | 10% PG | 5% DGME | |
| | and 18% | | | | |
| | K₂HPO₄ | | | | |
| 14 | 3.35% NaOH | 1.00% C-99 | 10% PG | | |
| | and 18% | | | | |
| | K₂HPO₄ | | | | |
| 15 | 3.35% NaOH | 1.00% C-99 | | 5% DGME | |
| | and 18% | | | | |
| | K₂HPO₄ | | | | |

Suspension tests were run on various formulations of Use Solutions of 2 part chemistry based on Part B formulations from Table 6 against G. stearo. Results are shown in Table 7.

TABLE 7

| Part B Formulation Tested (ID from Table 6) | CFU Count After |
|---|---|
| 1 | 45 |
| 2 | 99 |
| 3 | 52 |
| 4 | 41 |
| 5 | 188 |
| 7 | 150 |
| 6 | 4.5 |

Note:
The test was done at 3-minute contact time and 20° C.

Table 8 shows the suspension test results for Use Solutions of 2 part chemistry based on Part B formulations from Table 6 against B. subtilis.

TABLE 8

| Part B Formulation Tested (ID from Table 6) | CFU Count After |
|---|---|
| 1 | 273 |
| 2 | 332 |
| 3 | 158 |
| 4 | 84.5 |
| 7 | 264 |

Note:
The test was done at 4-minute contact time and 20° C.

Results in Tables 7 and 8 indicated that EDTA had negative affect on micro efficacy. The presence of diethylene glycol monoethyl ether and propylene glycol helped improved the micro efficacy of the new 2 part chemistry. Formula 1, 3, and 4 showed the most promising results for micro efficacy.

Another set of suspension tests were run to further evaluate the Part B formulation as well as to confirm the results obtained from in Tables 7 and 8. The results are shown in Table 9.

Table 9 shows the suspension test results for the Use Solutions utilizing the Part B formulations from Table 6 against B. subtilis.

TABLE 9

| Part B | H₂O₂ (ppm) of | PAA (ppm) of | pH of | CFU Count |
|---|---|---|---|---|
| Formula ID | Use Soln | Use Soln | Use Soln | After |
| 1 | 4113 | 2035 | 6.42 | TNTC |
| 3 | 4082 | 1969 | 6.55 | TNTC |
| 4 | 4290 | 2070 | 6.50 | TNTC |
| 7 | 9030 | 1979 | 4.22 | TNTC |
| 9 | 4033 | 1972 | 6.44 | TNTC |
| 11 | 4106 | 1987 | 6.46 | TNTC |
| 12 | 4493 | 2062 | 6.40 | 140 |
| 13 | 4476 | 2067 | 6.35 | 245 |

Note:
Test was done at 4 minutes contact time and 20° C. with PAA concentration for all testing at 2000 ppm PAA.

The results indicated that Formulation 12 and 13 showed the best feasible efficacy from the micro suspension test results.

A micro suspension test was performed on Use Solutions utilizing Formulas 11, 13, and 14 from Table 6 for Part B in comparison to Rapicide PA Double dose at different contact times against B. subtilis at 30° C. The results are shown in Table 10.

TABLE 10

| | CFU Count After | | | |
|---|---|---|---|---|
| Contact | Rapicide | Use Soln using | Use Soln using | Use Soln using |
| Time | PA Double | Part B Formula | Part B Formula | Part B Formula |
| (min) | Dose | 11 | 13 | 14 |
| 0.5 | TNTC | TNTC | TNTC | TNTC |
| 1 | TNTC | TNTC | TNTC | TNTC |
| 2 | 118 | 27 | 30 | 14 |
| 3 | 0 | 0 | 0 | 0 |
| 5 | 0 | 0 | 0 | 0 |
| 7 | 0 | 0 | 0 | 0 |
| 10 | 0 | 0 | 0 | 0 |

The results indicated that all three 2 part chemistry Use Solutions were better than Rapicide PA Double dose chemistry at 2 minutes contact time.

Table 11 shows the AOAC carrier results for Use Solutions having Part B formulations from Table 6 against B. subtilis loop

TABLE 11

| Use Solution Part B Formula from | |
|---|---|
| Table 6 (unless otherwise noted) | AOAC Carrier Test Results |
| Rapicide PA Double Dose | 0/20 |
| Formula 11 | 0/20 |
| Formula 13 | 0/20 |
| Formula 14 | 0/20 |

Note:
All 2 part chemistries were tested at 2000-2100 pm PAA and pH at 6.2-6.4. Rapicide PA Double Dose were tested at 2000 ppm PAA and pH 4.18.

The AOAC sporicidal carrier test against B. subtilis results showed that all three formulations tested were able to achieve complete kill.

Table 12 shows the AOAC carrier results for the 2 part chemistry Use Solution using Formula 14 for Part B from Table 6.

TABLE 12

| AOAC Carrier Micro-organism | Test Results |
|---|---|
| Bacillus subtilis | 0/60 |
| Clostridium sporogenes | 18/53 loops |
| | 0/7 penicylinder |

Note:
Formula 14 was tested with Use solution at 2069 ppm PAA and pH at 6.12.

The results showed survivors with C. sporogenes carrier test.

A further AOAC test was run to understand the affect the pH of the Use solution comparing Use Solutions having Part B formulas 13 and 14. The results are shown in Table 13.

TABLE 13

| Part B Formulation in the Use Solution | AOAC Carrier Test |
|---|---|
| (from Table 6) | Results |
| Formula 14 (pH 5) | 0/20 |
| Formula 14 (pH 6) | 1/20 |
| Formula 13 (pH 5) | 0/20 |
| Formula 13 (pH 6) | 0/20 |

Note:
All samples were tested at PAA concentration of 2000-2100 ppm PAA at 3 minutes contact time and 30° C.

Results showed that Formulas 13 and 14 had almost total kill. A pH less than 6 showed better micro efficacy against AOAC carrier testing.

The stability of Use Solution of the 2 part chemistry utilizing Formula 13 from Table 6 for Part B at 40° C. after mixing was run. The results are shown in Table 14.

TABLE 14
2 part stability at 40° C.

| Hours after Start | H₂O₂ (ppm) | PAA (ppm) |
|---|---|---|
| 0.00 | 5369 | 2534 |
| 1.33 | 5379 | 2497 |
| 2.50 | 5403 | 2450 |
| 4.00 | 5409 | 2382 |
| 7.00 | 5422 | 2296 |
| 22.50 | 5532 | 1907 |

Table 14 shows that for a desired MRC of PAA of 2000-2200 ppm, the Use Solution can be for up to 22.5 hours after mixing.

Additional corrosion tests were run in order to evaluate the effects of various components. Table 15 shows the corrosion rate of 2 part chemistry Use solutions with solubilizer and cleaner addition. Note: For formulation composition of Part B, please refer to Table 6.

TABLE 15

| Part B | | Corrosion Rate |
|---|---|---|
| Formulation ID | Description | Observed (mg/min) |
| 1 | Buffer and Anticorrosion only | 0.0129 |
| 3 | Buffer, anticorrosion and cleaner | 0.0098 |
| | only | |
| 4 | Buffer, anticorrosion and | 0.0092 |
| | solubilizer only | |
| 13 | Buffer, anticorrosion with both | 0.0076 |
| | cleaner and solubilizer | |

The results showed improvements on the corrosion rate when both cleaner and solubilizer were individually added to the buffer and anticorrosion formulation and further improvement when both components were added.

Suspension Test Comparison—A suspension test of Use Solutions with the microorganism B. subtilis was conducted in order to understand the micro efficacy of the addition of the solubilizer, cleaner or both. The results are shown in Table 16. For Part B formulation composition, please refer to Table 6. Note: “CFU” stands for “Colony Forming Unit” which is the measure of microorganism growth during a suspension test. The lower the CFU count the better efficacy the chemistry have against the microorganism. “TNTC” stands for “Too numerous to count” which is used when the chemistry isn't effective and too much growth was observed after testing.

Table 16 shows the microbiological efficacy suspension test against B. subtilis for the 2 part chemistry.

TABLE 16

| Part B | | |
|---|---|---|
| Formulation ID | | CFU |
| from Table 6 | Description | Count |
| 1 | Buffer and Anticorrosion only | TNTC |
| 3 | Buffer, anticorrosion and cleaner only | TNTC |
| 4 | Buffer, anticorrosion and solubilizer only | TNTC |
| 11 | Buffer and Anticorrosion only | TNTC |
| 13 | Buffer, anticorrosion with both cleaner and | 245 |
| | solubilizer | |

The results in Table 16 showed that countable results for the microbiological efficacy are obtained when both the cleaner and the solubilizer components were present. For the comparison study, it was also found that even though the Formulation 1 and 13 have slightly different buffer and anticorrosion composition, the results were still comparable based on the results of microbiological efficacy study where the different buffer and anticorrosion composition and/or components were tested.

Table 17 shows the microbiological efficacy suspension test against B. subtilis for the different buffer and anticorrosion formulation.

TABLE 17

| Part B | | |
|---|---|---|
| Formulation ID | | |
| from Table 6 | Formulation Composition | CFU Count |
| 1 | 4.5% NaOH + 14.2% K₂HPO₄ + 0.5% C- | TNTC |
| | 99 | |
| 9 | 21.2% K₃PO₄ + 1.0% C-99 | TNTC |
| 11 | 3.35% NaOH + 18% K₂HPO₄ + 1.0% C- | TNTC |
| | 99 | |

The results from Table 17 show that it did not matter which buffer composition or anticorrosion composition used. The suspension test showed no noticeable difference since the buffer and anticorrosion alone did not have any effect on the microbiological efficacy of the sterilant.

Table 18 shows the AOAC carrier test results for 2 Part Chemistry with Part B Formulation ID 13 from Table 6.

TABLE 18
AOAC carrier test results:

| | |
|---|---|
| Bacillus subtilis Loops | 0/60 |
| C. Sporogenes Loops | 0/60 |

Note:
Testing was done at 5 minutes contact time and 30° C. with chemistry at 2043 ppm PAA and pH 5.46.

Table 18 shows there were 0 survivors of the carriers.

The 2 part chemistry was subjected to viricidal testing to determine the contact time needed for sterilization. A film of virus, dried on a glass surface, was exposed to a 2.00 mL aliquot of the Use Solution for both a 5 minute and 10 minute exposure time at 30° C. Following each exposure time, the virucidal and cytotoxic activities were removed from the virus test substance mixtures utilizing a Sephadex gel column, and the mixtures were assayed for viral infectivity by an accepted assay method. Appropriate virus, test substance cytotoxicity, and neutralization controls were run concurrently.

Table 19 shows the virucidal test results for 2 part chemistry Use Solution with Formulation ID 13 from Table 6 for part B.

TABLE 19
Virucidal test results:

| Condition Tested | Results |
|---|---|
| 5 min contact time and 30° C. | 3.75 log red |
| 6 min contact time and 30° C. | ≥5.25 log red |
| 7 min contact time and 30° C. | ≥4.50 log red |
| 8 min contact time and 30° C. | ≥4.50 log red |
| 10 min contact time and 30° C. | ≥4.50 log red |

Note
Testing was against Poliovirus Type 1. Criteria is 4 log reductions or higher.

The results showed that for virucidal efficacy, the 2 part chemistry was able to pass at 6 minutes contact time and 30±2° C. Both the AOAC carrier test and virucidal test showed that the 2 part chemistry Use Solutions utilizing Formulation 13 from Table 6 for Part B was able to achieve good sterilization at a low temperature.

Although the present invention has been described with reference to preferred embodiments, persons skilled in the art will recognize that changes may be made in form and detail without departing from the spirit and scope of the invention. All references cited throughout the specification, including those in the background, are incorporated herein in their entirety. Those skilled in the art will recognize, or be able to ascertain, using no more than routine experimentation, many equivalents to specific embodiments of the invention described specifically herein. Such equivalents are intended to be encompassed in the scope of the following claims.

1. A two part liquid sterilant composition comprising components (A) and (B) to be mixed to yield an aqueous composition, wherein component (A) comprises hydrogen peroxide, acetic acid and/or peracetic acid and a polymeric sulfonic acid resin based chelator; and component (B) comprises a buffer, an anticorrosive agent, a solubilizer and a cleaner, wherein when component (A) and component (B) are combined, form a liquid sterilant.

2. (canceled)

3. (canceled)

4. (canceled)

5. The two part liquid composition of claim 21, wherein the polymeric sulfonic resin functionalized resin is a divinyl benzene/styrene copolymer, a perfluorosulfonic acid resin or a polymer containing a 2-acrylamido-2-methylpropane sulfonic acid resin.

6. The two part liquid composition of claim 5, wherein the polymeric sulfonic resin functionalized resin chelator is present in a range of from about 0.25% to about 1% based on the total weight percent of the liquid sterilant.

7. The two part liquid composition of claim 1, wherein the buffer comprises sodium hydroxide and potassium phosphate dibasic.

8. The two part liquid composition of claim 7, wherein the sodium hydroxide is present in a range of from about 3% to about 4.5% based on the total weight percent of the liquid sterilant.

9. The two part liquid composition of claim 7, wherein the potassium phosphate dibasic is present in a range of from about 14% to about 18% based on the total weight percent of the liquid sterilant.

10. The two part liquid composition of claim 1, wherein the anticorrosive is benzotriazole.

11. (canceled)

12. The two part liquid composition of claim 1, wherein the solubilizer is propylene glycol.

13. The two part liquid composition claim 12, wherein the propylene glycol is present in a range of from about 9.5% to about 10.5% based on the total weight percent of the liquid sterilant.

14. The two part liquid composition of claim 1, wherein the cleaner is diethylene glycol mono-ethyl ether.

15. (canceled)

16. The two part liquid composition of claim 1, wherein the polymeric sulfonic acid resin based chelator is present in a range of from about 0.1% to about 1%, the buffer is present in a range of from about 3% to about 21%, the anticorrosive is present in a range of from about 0.25% to about 1%, the solubilizer is present in a range of from about 9.5% to about 10.5% and the cleaner is present in a range of from about 4.75% to about 5.25% based on the total weight percent of the liquid sterilant.

17. The two part liquid composition of claim 1, wherein the polymeric sulfonic acid resin based chelator is present in a range of from about 0.1% to about 1%, sodium hydroxide is present in a range of from about 3.35% to about 4.5%, potassium phosphate dibasic is present in a range of from about 14.2% to about 18%, benzotriazole is present in a range of from about 0.25% to about 1%, propylene glycol is present in a range of from about 9.5% to about 10.5% and diethylene glycol mono-ethyl ether is present in a range of from about 4.75% to about 5.25% based on the total weight percent of the liquid sterilant.

18. The two part liquid composition of claim 17, wherein the polymeric sulfonic acid resin based chelator is present at 0.1%, the sodium hydroxide is present at 3.35%, the potassium phosphate dibasic is present at 18%, the benzotriazole is present at 1%, the propylene glycol is present 10% and the diethylene glycol mono-ethyl ether is present 5% based on the total weight percent of the liquid sterilant.

19. The two part liquid composition of claim 1, wherein the composition does not include sulfuric acid.

20. The two part liquid composition of claim 1, wherein the composition does not include EDTA.

21. The two part liquid composition of claim 1, wherein the component (A), component (B) and water are mixed in a ratio of 0.5:0.5:49 to 5:5:40 to provide the liquid sterilant.

22. (canceled)

23. The two part liquid composition of claim 1, wherein the liquid sterilant has a peracetic acid concentration of from about 2000 ppm PAA to about 3000 ppm PAA.

24. The two part liquid composition of claim 1, wherein the liquid sterilant has a pH of greater than 5 to and less than 6.5.

25. (canceled)

26. (canceled)

27. A method to clean and disinfect or sterilize a substrate contaminated with microorganisms comprising the steps of;
providing a two part liquid composition, comprising components (A) and (B) to be mixed to yield an aqueous composition, wherein component (A) comprises hydrogen peroxide, acetic acid and/or peracetic acid and a polymeric sulfonic acid resin based chelator; and component (B) comprises a buffer, an anticorrosive agent, a solubilizer and a cleaner, wherein when component (A) and component (B) are combined, form a liquid sterilant; and
contacting the substrate with the liquid sterilant to clean and disinfect or sterilize the substrate.

28. (canceled)

29. (canceled)

30. (canceled)

31. (canceled)

32. A kit comprising:
container (A) and container (B), wherein container A contains hydrogen peroxide, acetic acid and/or peracetic acid and a polymeric sulfonic acid resin based chelator and container B contains a buffer, an anticorrosive agent, a solubilizer and a cleaner;
instructions for combining contents of containers (A) and (B) to provide a liquid sterilant composition; and
instructions for contacting a substrate contaminated with microorganisms using the liquid sterilant composition.
